(19) United States
(12) Patent Application Publication
(10) Pub. No.: US 2018/0110432 A1
(43) Pub. Date: Apr. 26, 2018

(54) METHOD FOR AUTOMATIC DETECTION OF CHF AND AF WITH SHORT RR INTERVAL TIME SERIES USING ELECTROCARDIOGRAM

(71) Applicant: Soonchunhyang University Industry Academy Cooperation Foundation, Chungcheongnam-do (KR)

(72) Inventors: Yun-Young NAM, Chungcheongnam-do (KR); Jin-Seok LEE, Daejeon (KR)

(21) Appl. No.: 15/534,919

(22) PCT Filed: Dec. 9, 2015
(86) PCT No.: PCT/KR2015/013424

§ 371 Date: Jun. 9, 2017

(30) Foreign Application Priority Data
Dec. 12, 2014 (KR) 10-2014-0179413

(51) Int. Cl. A61B 5/046; A61B 5/04

(52) U.S. Cl. CPC A61B 5/046; A61B 5/04012

(57) ABSTRACT

A method for automatic detection of CHF and AF with 12-bit RR interval time series using an electrocardiogram by using normalized RMSSD, sample entropy, and Shannon entropy, includes: (a) testing the electrocardiogram according to time from a subject; (b) calculating normalized RMSSD of data obtained at step (a); (c) calculating sample entropy value of the data obtained at step (a); (d) calculating Shannon entropy value for each of sections divided at step (a); and (e) comparing the normalized RMSSD, the sample entropy, and the Shannon entropy values calculated at steps (a),(b), and (c), and detecting CHF and AF.

### BACKGROUND

The present invention relates to a method for automatic detection of CHF and AF with short RR interval time series using an electrocardiogram with normalized RMSSD, sample entropy, and Shannon entropy.

Atrial fibrillation (hereinafter, abbreviated as ‘AF’) and congestive heart failure (hereinafter, abbreviated as ‘CHF’) are fatal diseases that is increasingly widespread and costly, and are related to morbidity rate and mortality rate.

AF is the most common persistent arrhythmia and morbidity rate. Currently, more than 2.3 million people have AF in the United States of America. Through a relation between a stroke and increased risk of a death, AF greatly affects life expectancy and quality of life. Although AF treatment strategies can be used, clinicians and researchers face main task of dealing with frequent convulsions and asymptomatic features of AF, especially in the early stages. Even a simple episode of AF is associated with an adverse health outcome, and thus it is urgently required to develop a method of accurately detecting paroxysmal AF.

Likewise, CHF becomes an increasingly more common disease according to population age. CHF affects almost 5 million people, and takes the lives of 200,000 people every year in the U.S.A. Hypertension, myocardial infarction, obesity, diabetes, renal failure, anemia, disorder of cardiac valves, depressive disorder cause risk of CHF, however, early detection of CHF increases therapeutic effects of CHF.

For this reason, AF and CHF detection technologies are emphasized. Electrocardiography (surface 12-leads ECG) is one of most useful tests for diagnosis and prognosis of AF and CHF patients. Electrocardiography is regarded as one of useful tests that provide data on the heartbeat, and then diagnoses and prognoses morphological changes in ECG curve's components.

However, even through standard 12-lead electrocardiography and symptom monitoring are excellent, AF and CHF monitoring methods cannot sufficiently analyze or identify bit-to-bit data every day that identifies the presence of AF and CHF, and cannot identify and distinguish AF, CHF and normal sinus rhythm (hereinafter, abbreviated as ‘NSR’).

Therefore, it is required to develop an accurate and comprehensive automatic detection algorithm by using a standard ECG recorder. In this work, by using an ECG record that is commercially available and clinically applicable, we developed a sensitive, real-time realization bit-to-bit detection algorithm for atrial fibrillation (AF), congestive heart failure (CHF), and normal sinus rhythm (NSR).

For last 10 years, many algorithms have been proposed to detect AF by using RR interval (RRI) variability. Approach is based on the characteristics of AF as remarkably increasing bit-to-bit changes and random sequence of complexity RRI series. Most algorithms show high sensitivity, specificity, and accuracy of up to 97%. Also, recently, this method has been applied to smartphone applications. Actually, a built-in camera lens may be used to detect AF based on the function of recoding fingertip pulsatile photoplethysmogram signals.

Also, CHF detection using RRI time series has been studied. In previous studies, cardiac confusion occurs more often in a healthy heart, thus a decrease in this confusion may indicate CHF.

In fact, while AF has high variability and complexity, CHF has extremely low variability and complexity in RRI time series. However, most conventional studies used relatively long RRI time series, and thus it was impossible to analyze bit-to-bit data to identify the presence of CHF every day.

(Patent Document 1) Korean Patent No. 10-0493714 (registration date: May 26, 2005)

### SUMMARY OF THE INVENTION

The present invention is intended to propose a method for automatic detection of CHF and AF with short RR interval time series using an electrocardiogram by calculating normalized RMSSD, sample entropy, and Shannon entropy.

In order to accomplish the above object, the present invention provides a method for automatic detection of CHF and AF with short RR interval time series using an electrocardiogram, the method including: (a) testing the electrocardiogram according to time from a subject; (b) identifying 12-bit RR interval (RRI) time series from data obtained at the step (a); (c) calculating normalized RMSSD of data identified at the step (b); (d) calculating sample entropy value of data identified at the step (b); (e) calculating Shannon entropy value for each of sections identified and divided at the step (b); (f) comparing the normalized RMSSD, the sample entropy value, and the Shannon entropy value calculated at the steps (c), (d), and (e), and detecting CHF when satisfying Normalized RMSSD≤TH_{RM}^{C}, SampEn≤TH_{SA}^{C}, ShEn≤TH_{SE}^{C}or Normalized RMSSD≤TH_{RM}^{C}, SampEn≤TH_{SA}^{C}, and detecting AF when satisfying Normalized RMSSD≥TH_{RM}^{A}, SampEn≥TH_{SA}^{A}, ShEn≥TH_{SE}^{A} or Normalized RMSSD≥TH_{RM}^{A}, SampEn≥TH_{SA}^{A}.

Preferably, detection of AF or CHF may identify a threshold value by using an ROC curve. Values of overall sensitivity, specificity, and accuracy may be calculated by being averaged from results of AF and CHF. In MIT-BIH AF, MIT-BIH NSR, and BIDMC CHF databases, a number of FP (false positives) and FN(false negatives) may be identified, and in CHF RRI database, a number of TP (true positives), TN (true negatives), FP (false positives), and FN (false negatives) may be identified so as to identify the normalized RMSSD, the sample entropy, and the Shannon entropy. Sensitivity TP/(TP+FN), specificity TN/(TN+FP), and accuracy (TP+TN)/(TP+TN+FP+FN) for AF CHF may be calculated.

According to the present invention, the method for automatic detection of CHF and AF with short RR interval time series using an electrocardiogram can detect CHF and AF with the best accuracy. That is, it is highly effective in overall sensitivity, specificity, and accuracy of AF and CHF.

### BRIEF DESCRIPTION OF DRAWINGS

FIG. 1 is a graph showing a distribution of statistical values of normalized RMSSD for each piece of data by using 16 bits.

FIG. 2 is a graph showing a distribution of statistical values of sample entropy for each piece of data by using 16 bits.

FIG. 3 is a graph showing a distribution of statistical values of sample entropy for each piece of data by using 16 bits.

FIG. 4 is a graph showing ROC curve based normalized RMSSD for detecting AF and CHF (the left for AF, and the right for CHF).

FIG. 5 is a graph showing ROC curve based sample entropy for AF and CHF (the left for AF, and the right for CH).

FIG. 6 is a graph showing ROC curve based Shannon entropy for AF and CHF (the left for AF, and the right for CH).

### DETAILED DESCRIPTION OF THE INVENTION

According to the present invention, a method for automatic detection of CHF and AF with short RR interval time series using an electrocardiogram includes:

(a) testing the electrocardiogram according to time from a subject, (b) calculating normalized RMSSD of data obtained at step (a), (c) calculating sample entropy value of the data obtained at step (a), (d) calculating Shannon entropy value for each of sections divided at step (a), and (f) comparing the normalized RMSSD, the sample entropy, Shannon entropy values calculated at steps (a),(b), and (c), and detecting CHF in a case of Normalized RMSSD≤TH_{RM}^{C}, SampEn≤TH_{SA} ^{C} and/or ShEn≤TH_{SE}^{C}, and detecting AF in a case of Normalized RMSSD≥TH_{RM}^{A}, SampEn≥TH_{SA}^{A} and/or ShEn>TH_{SE}^{A}.

Hereinafter, the present invention will be described in detail with reference to the accompanying drawings.

Experimental Condition

Five databases such as MIT-BIH AF, MIT-BIH NSR, BIDMC CHF, CHF RRI, and 24-hour clinical CHF databases were used at Wonkwang University School of Medicine (South Korea). RR interval time series was used in all databases. The MIT-BIH AF database contains 25 ECG signals in which there are episodes of 299 AF. Each ECG record is approximately 10 hours. Incorrect AF annotations are excluded due to 4936 and 5091 data. The MIT-BIH NSR database contains 18 ECG records, and each ECG record is approximately 24 hours. NSR data do not contain AF and CHF episodes. The BIDMC CHF database contains serious CHF (NYHA class III-IV) 15 ECG records. A personal record is approximately 20 hours of the period. While treating the patient, the doctor needs to classify in the severity of the patient's condition. The most widely used classification system is New York Heart Association (NYHA) classification system. According to this system, classification ranges from CHF IV (severe) to I (mild). The CHF RRI database contains CHF (NYHA I-III) 29 ECG records. Personal recoding is approximately 20 to 24 hours.

Data sets 02 and 06 in BIDMC CHF. In CHF RRI, 207, 213, 214, and 221 are excluded since accurate detection may be interrupted due to extremely high PAC and/or PVC frequency (equal to greater than 50%). Clinical AF and CHF (congestive heart failure) databases monitor 24-hour Holter data, and monitor 14 AF, 14 CHF (NYHA III-IV), and 14 NSR collected from 42 subjects by using GE Prophet Light Holter.

After obtaining heart rate and blood pressure as well as basic clinical, demographic, laboratory, electrophysiological variables, education research staff extracted the medical records of participants. Data is obtained at 125 samples per second with 10-bit resolution. CHF, AF, and NSR data annotates all heart beats. Extracted RR intervals are analyzed by using 2012b of MAT LAB.

In the present invention, short (12-bit) RRI time series are used with normalized RMSSD, sample entropy, and Shannon entropy values. The RRI time series segment statistical value corresponds to the center bit of the segment (for example, 8-bit). The procedure is repeated by shifting the bit per RRI time series segment. In this way, classification is performed into bit-to-bit of AF, CHF, and NSR. CHF including premature beats or NSR can use RRI time series. Also, missing or false peak detection is monitored as incorrect classification results of CHF, NSR, and AF. In order to reduce influence by premature beats, the two longest RR values and the two shortest RR values in the segment are considered as errors and removed.

### Calculation Step of Normalized RMSSD

RMSSD is used in quantifying beat change variability. AF shows higher variability than CHF and NSR, and CHF shows lower variability than AF and NSR. Thus, it is expected to distinguish CHF, AF, and NSR by RMSSD.

Normalized RMSSD was normalized by dividing RMSSD with a mean value of RR interval time series. It is calculated by the following formula (1). Here, segment rr(i) of RR interval time series includes 1.

$$normalize RMSSD = \sqrt{\frac{1}{l - 1} {\sum }_{i = 1}^{l - 1} {( rr ( i + 1 ) - rr ( i ) )}^{2}} \qquad [ Formula 1 ]$$

Accordingly, based on the MIT-BIH AF, MIT-BIH NSR, BIDMC CHF, and CHF RRI databases, normalized RMSSD of 12-bit RRI time may be calculated. Each segment is moved by one bit through the entire databases.

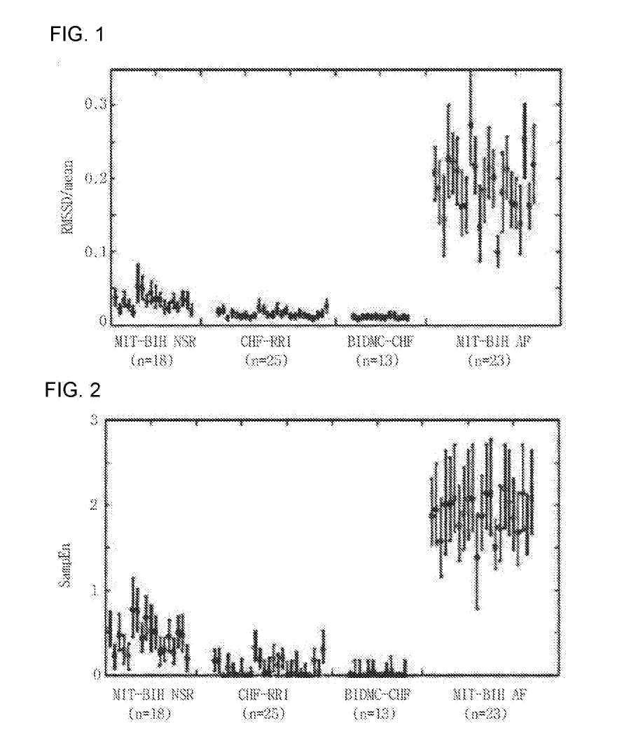

FIG. 1 shows a distribution of statistical values of normalized RMSSD values of 18 NSR of the MIT-BIH NSR database, 25 CHF of the CHF RRI database, 13 CHF of the BIDMC-CHF database, and 23 AF of the MIT-BIH AF database. All statistical values are indicated as 12-bit RRI time series after removing outliers. Here, the upper, middle, and lower points of the bar in the graph respectively indicate 75, 50, and 25%.

As shown in FIG. 1, RMSSD mean value for AF is high, and is almost distinguished from those of CHF and NSR databases. Also, normalized RMSSD value for CHF is extremely low and is almost distinguished from those of AF and NSR databases.

In addition, table 1 shows mean, median, and interquartile range (IQR) values for NSR, CHF (NYHA CHF (NYHA III-IV), and AF that use normalized RMSSD. Here, 12-bit RRI time series normalized RMSSD value is based on the MIT-BIH AF, MIT-BIH NSR, BIDMC CHF, CHF RRI databases.

TABLE 1

| | Classification | Mean ± SD | Median | IQR |
|---|---|---|---|---|
| RMSSD/Mean | AF | 0.1907 ± 0.0783 | 0.1858 | 0.1090 |
| | NSR | 0.0354 ± 0.0274 | 0.0297 | 0.0221 |
| | CHF | 0.0210 ± 0.0509 | 0.0137 | 0.0096 |
| | (NYHAI~III) | | | |
| | CHF | 0.0135 ± 0.0287 | 0.0102 | 0.0056 |
| | (NYHAIII~IV) | | | |

As shown in table 1, the mean value of CHF (NYHA I-III) was 9.08 times lower than that of AF, and was 1.69 times lower than that of NSR. The mean value of CHF (NYHA III-IV) was 14.13 times lower than that of AF, and was 2.62 times lower than that of NSR. The median value of CHF (NYHA I-III) was 13.56 times lower than that of AF, and was 2.17 times lower than that of NSR. The median value of CHF (NYHA III-IV) was 18.21 times lower than that of AF, and was 2.92 times lower than that of NSR. The IQR value of CHF (NYHA I-III) was 11.35 times lower than that of AF, and was 2.30 times lower than that of NSR. The IQR value of CHF (NYHA III-IV) was 19.46 times lower than that of AF (atrial fibrillation), and was 3.96 times lower than that of NSR (normal sinus rhythm).

### Calculation Step of Sample Entropy

Sample entropy is a negative natural log of conditional probability estimation where a subordinate record series matching for each point match at the next point within allowable error. Sample entropy is measured based on comparing patterns in time series to estimate complexity. This sample entropy is characterized by physiological time series analysis state information, and is successfully used as a complexity measured value in short-term biological signal studies.

Sample entropy divides RR interval time series (rr) into patterns of segment length 1 first, and is calculated by formula (2).

$$SampEn ( l , r ) = - log \frac{{\sum }_{i = 1}^{N - 1} {N}_{i} ( l + 1 , r )}{{\sum }_{i = 1}^{N - 1} {N}_{i} ( l , r )} \qquad [ Formula 2 ]$$

Here, Nᵢ (1,r) is the number of patterns of length 1 found at a distance less than the threshold value r in RR interval time series of length 1. Nᵢ (1+1,r) is the number of patterns of length 1+1 found at a distance less than the threshold value r in RR interval time series of length 1+1. The sum of (2) expands to all patterns of length 1+1, and distance is evaluated as Euclidean norm.

Here, based on the same MIT-BIH AF, MIT-BIH NSR, BIDMC CHF, and CHF RRI databases, the sample entropy of 12-bit RRI time series shifted by all bits is calculated.

FIG. 2 shows a distribution of statistical values of SampEn distribution value based on the same database as the normalized RMSSD. Here, the upper, middle, and lower points of the bar in the graph respectively indicate 75, 50, and 25%.

As shown in FIG. 2, similar to normalized RMSSD, the sample entropy value for AF is higher than those of CHF and NSR databases, and is distinguished therefrom. Also, the sample entropy value for CHF is low, and is distinguished from those of AF and NSR databases.

In addition, table 2 shows mean, median, and interquartile range (IQR) values for NSR, CHF (NYHA CHF (NYHA III-IV), and AF that use sample entropy. Here, 12-bit RRI time series sample entropy value is based on the MIT-BIH AF, MIT-BIH NSR, BIDMC CHF, and CHF RRI databases.

TABLE 2

| | Classification | Mean ± SD | Median | IQR |
|---|---|---|---|---|
| SampEn | AF | 2.0002 ± 0.6771 | 1.8971 | 0.9808 |
| | NSR | 0.4705 ± 0.3520 | 0.4055 | 0.4305 |
| | CHF | 0.1467 ± 0.2123 | 0.0488 | 0.2144 |
| | (NYHAI~III) | | | |
| | CHF | 0.0623 ± 0.1316 | 0.0000 | 0.0783 |
| | (NYHAIII~IV) | | | |

As shown in table 2, the mean value of CHF (NYHA I-III) was 13.63 times lower than that of AF, and was 3.20 times lower than that of NSR. The mean value of CHF (NYHA III-IV) was 32.11 times lower than that of AF, and was 7.55 times lower than that of NSR. The median value of CHF (NYHA I-III) was 38.88 times lower than that of AF, and was 8.31 times lower than that of NSR. The median value of CHF (NYHA III-IV) was zero, but those of AF and NSR were respectively 1.8971 and 0.4055. The IQR value of CHF (NYHA I-III) was 4.57 times lower than that of AF, and was 2.01 times lower than that of NSR. The IQR value of CHF (NYHA III-IV) was 12.53 times lower than that of AF, and was 5.50 times lower than that of NSR.

### Calculation Step of Shannon Entropy

Shannon entropy provides quantitative measurement of uncertainty for random variables. The Shannon entropy quantifies the feasibility of patterns that represent regularity during some period of data. For example, it is expected that a sine wave signal where an arbitrary white noise signal is simple will have a value close to zero. In contrast, Shannon entropy is expected to have a value close to one. From these characteristics, the Shannon entropy of CHF and NSR is expected to be lower than AF. Also, the Shannon entropy of CHF is expected to be lower than NSR. A histogram of RR interval time series segment for calculating Shannon entropy is obtained. RR values are classified into bins having uniform intervals, and the limit is limited to the shortest and the longest RR values. Probability distribution P(n) calculates each bin by using the number of bits, and a bin is divided by the total number of segment beats. Shannon entropy is calculated by the following formula (3).

$$ShEn = - {\sum }_{n = 1}^{{N}_{bm}} P ( n ) \frac{log ( p ( n ) )}{{log ( \frac{1}{{N}_{bin}} )}^{′}} \qquad [ Formula 3 ]$$

Here, N_{bin} is the number of bins.

Here, based on the same MIT-BIH AF, MIT-BIH NSR, BIDMC CHF, and CHF RRI databases, Shannon entropy in 12-bit RRI time series shifted by all bits was calculated.

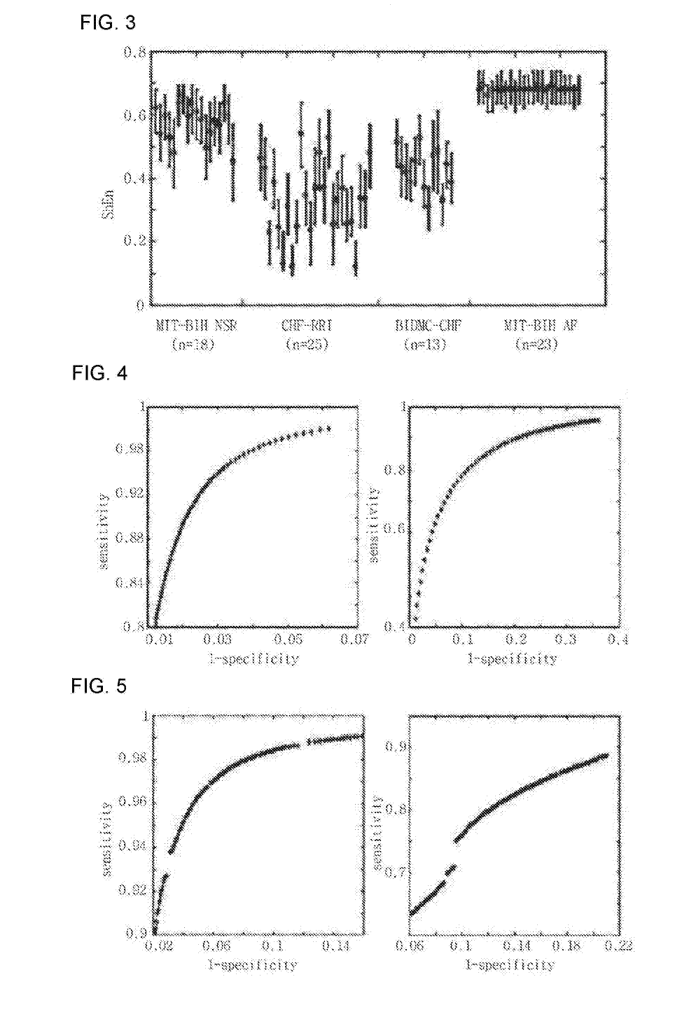

FIG. 3 shows a distribution of statistical values of Shannon entropy distribution value based on the same database as normalized RMSSD and sample entropy. Here, the upper, middle, and lower points of the bar in the graph respectively indicate 75, 50, and 25%.

As shown in FIG. 3, similar to the sample entropy, Shannon entropy value or AF is higher than those of normalized RMSSD, CHF, and NSR databases, and is distinguished therefrom. Also, Shannon entropy value for CHF is low, and is distinguished from those of AF and NSR databases.

Table 3 shows mean, median, and interquartile range (IQR) values for NSR, CHF (NYHA CHF (NYHA III-IV), and AF that use Shannon entropy. Here, 12-bit RRI time series Shannon entropy value was based on the MIT-BIH AF, MIT-BIH NSR, BIDMC CHF, and CHF RRI databases.

TABLE 3

| | Classification | Mean ± SD | Median | IQR |
|---|---|---|---|---|
| ShEn | AF | 0.6762 ± 0.0634 | 0.6808 | 0.0991 |
| | NSR | 0.5605 ± 0.1319 | 0.5818 | 0.1722 |
| | CHF | 0.3296 ± 0.1654 | 0.3247 | 0.2398 |
| | (NYHAI~III) | | | |
| | CHF | 0.4188 ± 0.1340 | 0.4151 | 0.1824 |
| | (NYHAIII~IV) | | | |

As shown in Table 3, the mean value of CHF (NYHA I-III) was 2.05 times lower than that of AF, and was 1.70 times lower than that of NSR. The mean value of CHF (NYHA III-IV) was 1.61 times lower than that of AF, and was 1.34 times lower than that of NSR. The median value of CHF (NYHA I-III) was 2.10 times lower than that of AF, and was 1.79 times lower than that of NSR. The median value of CHF (NYHA III-IV) was 1.64 times lower than that of AF, and was 1.40 times lower than that of NSR.

### CHF or AF Detection Step

CHF and AF detection is based on a threshold value of normalized RMSSD (RM), sample entropy (SE), and Shannon entropy (SA). CHF is TH _{RM}^{C}, TH_{SE}^{C} and TH_{SA}^{C}, AF is TH_{RM}^{A}, TH_{SE}^{A} and TH_{SA}^{A}, and TH_{RM}^{C}<TH_{RM}^{A}, TH_{SA}^{C}<TH_{SA}^{A}, TH_{SE}^{C}<TH_{SE}^{A}.

The detection condition of CHF satisfies Normalized RMSSD≤TH_{RM}^{C}, SampEN≤TH_{SA}^{C}, ShEn≤TH_{SE}^{C}, or Normalized RMSSD≤TH_{RM}^{C}, SampEn≤TH_{SA}^{C}. The detection condition of AF satisfies Normalized RMSSD≥TH_{RM}^{A}, SampEN≥TH_{SA}^{A}, ShEn≥TH_{SE}^{A}, or Normalized RMSSD≥TH_{RM}^{A}, SampEN≥TH_{SA}^{A}. Here, sample entropy and Shannon entropy use r=0.06 and N_{bin}=16.

For selection of each optimum threshold value, each possible threshold value was searched with interval increment as follows.

1) TH_{RM}^{C} was changed from 0.001 to 0.050 with an interval of 0.001;

2) TH_{SA}^{C} was changed from 0.100 to 0.200 with an interval of 0.001;

3) TH_{SE}^{C} was changed from 0.200 to 0.800 with an interval of 0.001;

4) TH_{RM}^{A} was changed from 0.010 to 0.150 with an interval of 0.001;

5) TH_{SA}^{A} was changed from 0.800 to 1.500 with an interval of 0.001;

6) TH_{SE}^{A} was changed from 0.500 to 1.000 with an interval of 0.001.

First, performance of each method was investigated.

In the MIT-BIH AF, MIT-BIH NSR, and BIDMC CHF databases, the number of FP (false positives) and FN (false negatives) is identified. In the CHF RRI database, the number of TP (true positives), TN (true negatives), FP (false positives), and FN (false negatives) is identified. Consequently, the number of normalized RMSSD, sample entropy, and Shannon entropy is identified.

Each of TP, TN, FP, FN, and each set is calculated for AF and CHF. Next, sensitivity TP/(TP+FN), specificity TN/(TN+FP), accuracy (TP+TN)/(TP+TN+FP+FN) for AF and CHF are calculated.

With each statistical method, in order to detect AF and CHF, threshold values are identified by using ROC curves. The values of overall sensitivity, specificity, and accuracy are calculated by being averaged from the results of AF and CHF.

CHF is detected in a case of Normalized RMSSD≤TH_{RM}^{C}, SampEN≤TH_{SA}^{C}, ShEn≤TH_{SE}^{C}, and AF is detected in a case of Normalized RMSSD≥TH_{RM}^{A}, SampEN≥TH_{SA}^{A}, ShEn≥TH_{SE}^{A}. In order to detect CHF by using multivariate analysis, first, TH_{RM}^{C}based ROC curve for fixing TH_{SE}^{C}=200 and TH_{SA}^{C}=100 was obtained.

Next, with an interval of 0.001, TH_{SA}^{C} was increased from 0.100 to 0.200, and TH_{RM}^{C} based ROC curve was obtained. Repeatedly, with an interval of 0.001, 0.200 was increased to 0.800. From the obtained ROC curve, the ROC curve providing the highest value in the ROC region was found. Similarly, ROC analysis was performed for detection of AF. Table 4 shows sensitivity, specificity, and accuracy values to which a combined statistical method and a single statistical method for AF are applied. Table 5 shows sensitivity, specificity, and accuracy values to which a combined statistical method and a single statistical method for CHF are applied.

TABLE 4

| | Classi- | | | | |
|---|---|---|---|---|---|
| | fication | sensitivity | specificity | accuracy | Threshold |
| Single | Normalized | 0.9522 | 0.9649 | 0.9607 | 0.075 |
| Methods | RMSSD | | | | |
| | SampEn | 0.938 | 0.9684 | 0.9583 | 1.090 |
| | ShEn | 0.8336 | 0.7738 | 0.7771 | 0.620 |
| Combined | Normalized | 0.9506 | 0.9712 | 0.9643 | 0.072 |
| Methods | RMSSD & | | | | 0.820 |
| | SampEn | | | | |
| | All Three | 0.9495 | 0.9724 | 0.9648 | 0.054 |
| | Combination | | | | 0.740 |
| | | | | | 0.515 |

TABLE 5

| | Classi- | | | | |
|---|---|---|---|---|---|
| | fication | sensitivity | specificity | accuracy | Threshold |
| Single | Normalized | 0.7775 | 0.9012 | 0.8600 | 0.019 |
| Methods | RMSSD | | | | |
| | SampEn | 0.7718 | 0.8957 | 0.8544 | 0.184 |
| | ShEn | 0.7583 | 0.8790 | 0.8388 | 0.474 |
| Combined | Normalized | 0.7535 | 0.9112 | 0.8587 | 0.020 |
| Methods | RMSSD & | | | | 0.220 |
| | SampEn | | | | |
| | All Three | 0.7414 | 0.9124 | 0.8554 | 0.020 |
| | Combination | | | | 0.222 |
| | | | | | 0.600 |

FIG. 4 shows ROC curve based normalized RMSSD for AF and CHF detection. The left is for AF, and the right is for CHF.

As described above, when the threshold value is 0.075 for AF detection, sensitivity, specificity, and accuracy respectively have 0.9522, 0.9649, and 0.9607. When the threshold value is 0.0190 for CHF detection, sensitivity, specificity, and accuracy respectively have 0.7775, 0.9012, and 0.8600. Overall sensitivity, specificity, and accuracy of AF and CHF were respectively 0.8649, 0.9331, and 0.9104.

As expected, when the threshold value was 0.0190, detection rate for CHF (NYHA I-III) was 0.7312, and detection rate for CHF (NYHA III-IV) was 0.9113. When a patient has more severe CHF, detection rate is high.

FIG. 5 shows ROC curve based sample entropy for AF and CHF. The left is for AF, and the right is for CH. When the threshold value is 1.090 for AF detection, sensitivity, specificity, and accuracy respectively have 0.9380, 0.9684, and 0.9583. When the threshold value is 0.184 for CHF detection, sensitivity, specificity, and accuracy respectively have 0.7718, 0.8957, and 0.8544. Overall sensitivity, specificity, and accuracy of AF and CHF were respectively 0.8549, 0.9321, and 0.9064. Also, when the threshold value was 0.184, detection rate for CHF (NYHA IIII) was 0.7134, and rate for CHF (NYHA III-IV) was 0.8902.

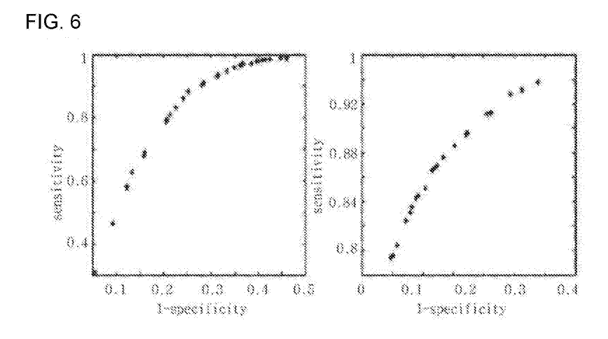

FIG. 6 shows ROC curve based Shannon entropy for AF and CHF. The left is for AF, and the right is for CHF. When the threshold value was 0.620 for AF detection, sensitivity, specificity, and accuracy were respectively 0.8336, 0.7738, and 0.7771. When the threshold value was 0.474 for CHF detection, sensitivity, specificity, and accuracy were respectively 0.7583, 0.8790, and 0.8388. Overall sensitivity, specificity, and accuracy of AF and CHF were respectively 0.7960, 0.8264, and 0.8587. Also, when the threshold value is 0.474, detection rate for CHF (NYHA I-III) is 0.7959, and rate for CHF (NYHA III-IV) is 0.6776.

As shown in FIGS. 1 to 3, by using the statistical method for distribution of each statistical value, normalized RMSSD and sample entropy are superior to Shannon entropy for CHF and AF detection. Normalized RMSSD had performance slightly better than that of sample entropy. In normalized RMSSD & sample entropy and ROC curve analysis, the best accuracy for AF detection was 0.9643 that was higher than normalized RMSSD by 0.0036 and sample entropy by 0.0060. In CHF detection, the best accuracy was 0.8587 that was lower than normalized RMSSD. Also, in a three combination method ROC curve analysis for AF and CHF, accuracy was 0.9648 and 0.8554.

### Clinical Data Result

In the same manner, AF and CHF detection is performed by using the entire 24-hour data as 12 bits, and data segment is shifted by one bit for the entire Holter recorder.

Consequently, by using TH_{RM}^{C}=0.019 and normalized RMSSD, sensitivity of 0.8104 and specificity of 0.9014 were obtained for CHF, sensitivity of 0.9514 for AF and specificity of 0.9798 in TH_{RM}^{A}=0.019 were obtained.

The result shows that it is applicable to normalized RMSSD and clinical data. Different two statistical methods of sample entropy and Shannon entropy provided high values similar to sensitivity and specificity compared to the MIT-BIH AF, MITBIH NSR, BIDMC CHF, and CHF RRI databases. However, the value was lower than that of normalized RMSSD. Clinical data results are indicated in tables 6 and 7.

Sensitivity, specificity, and accuracy values of a combined statistical method and a single statistical method for clinical data (N=42) set and AF.

TABLE 6

| | Classi- | | | | |
|---|---|---|---|---|---|
| | fication | sensitivity | specificity | accuracy | Threshold |
| Single | Normalized | 0.9514 | 0.9798 | 0.9656 | 0.075 |
| Methods | RMSSD | | | | |
| | SampEn | 0.9210 | 0.9690 | 0.9450 | 1.090 |
| | ShEn | 0.7976 | 0.7831 | 0.7904 | 0.620 |
| Combined | All Three | 0.9510 | 0.9643 | 0.9577 | 0.054 |
| Methods | Combination | | | | 0.740 |
| | | | | | 0.515 |

Sensitivity, specificity, and accuracy values of a combined statistical method and a single statistical method for clinical data (N=42) set and CHF (congestive heart failure).

TABLE 7

| | Classi- | | | | |
|---|---|---|---|---|---|
| | fication | sensitivity | specificity | accuracy | Threshold |
| Single | Normalized | 0.8104 | 0.9014 | 0.8559 | 0.019 |
| Methods | RMSSD | | | | |
| | SampEn | 0.7923 | 0.8981 | 0.8452 | 0.184 |
| | ShEn | 0.7644 | 0.8892 | 0.8268 | 0.474 |
| Combined | All Three | 0.7897 | 0.9001 | 0.8449 | 0.020 |
| Methods | Combination | | | | 0.222 |
| | | | | | 0.600 |

1. A method for automatic detection of CHF and AF with 12-bit RR interval time series using an electrocardiogram, the method comprising:
(a) testing the electrocardiogram according to time from a subject;
(b) identifying 12-bit RR interval (RRI) time series from data obtained at the step (a);
(c) calculating normalized RMSSD of data identified at the step (b) ;
(d) calculating sample entropy value of data identified at the step (b) ;
(e) calculating Shannon entropy value for each of sections identified and divided at the step (b);
(f) comparing the normalized RMSSD, the sample entropy value, and the Shannon entropy value calculated at the steps (c), (d), and (e), and detecting CHF when satisfying Normalized RMSSD≤TH_{RM}^{C}, SampEn≤TH_{SA}^{C}, ShEn≤TH_{SE}^{C}or Normalized RMSSD≤TH_{RM}^{C}, SampEn≤TH_{SA}^{C}, and detecting AF when satisfying Normalized RMSSD≥TH_{RM}^{A}, SampEn≥TH_{SA}^{A}, ShEn≥TH_{SE}^{A} or Normalized RMSSD≥TH_{RM}^{A}, SampEn≥TH_{SA}^{A}.

2. The method of claim 1, wherein the normalized RMSSD is calculated by formula (1)
$$normalize RMSSD = \sqrt{\frac{1}{l - 1} {\sum }_{i = 1}^{l - 1} {( rr ( i + 1 ) - rr ( i ) )}^{2}}$$
based on MIT-BIH AF, MIT-BIH NSR, BIDMC CHF, and CHF RRI databases, and
here, segment rr(i) of RR interval time series includes length (1).

3. The method of claim 1, wherein the sample entropy is calculated by formula (2)
$$SampEn ( l , r ) = - log \frac{{\sum }_{i = 1}^{N - 1} {N}_{i} ( l + 1 , r )}{{\sum }_{i = 1}^{N - 1} {N}_{i} ( l , r )}$$
based on MIT-BIH AF, MIT-BIH NSR, BIDMC CHF, and CHF RRI databases, and
here, Nᵢ (1,r) is a number of patterns of length 1 found at a distance less than a threshold value r in the RR interval time series of length 1, Nᵢ (1+1,r) is a number of patterns of length 1+1 found at a distance less than the threshold value r in the RR interval time series of length 1+1, a sum of formula (2) expands to all patterns of the length 1+1, and a distance is evaluated as Euclidean norm.

4. The method of claim 1, wherein the Shannon entropy is calculated by formula (3)
$$ShEn = - {\sum }_{n = 1}^{{N}_{bm}} P ( n ) \frac{log ( p ( n ) )}{{log ( \frac{1}{{N}_{bin}} )}^{′}}$$
based on MIT-BIH AF, MIT-BIH NSR, BIDMC CHF, and CHF RRI databases, and
here, N_{bin} is a number of bins.

5. The method of claim 1, wherein detection of AF or CHF identifies a threshold value by using an ROC curve,
values of overall sensitivity, specificity, and accuracy are calculated by being averaged from results of AF and CHF,
in MIT-BIH AF, MIT-BIH NSR, and BIDMC CHF databases, a number of FP (false positives) and FN (false negatives) is identified, and in CHF RRI database, a number of TP (true positives), TN (true negatives), FP (false positives), and FN (false negatives) is identified so as to identify the normalized RMSSD, the sample entropy, and the Shannon entropy, and
sensitivity TP/(TP+FN), specificity TN/(TN+FP), and accuracy (TP+TN)/(TP+TN+FP+FN) for AF or CHF are calculated.
